(19) United States
(12) Patent Application Publication
(10) Pub. No.: US 2021/0137877 A1
(43) Pub. Date: May 13, 2021

(54) PRODUCTS AND METHODS FOR USING CANNABIDIOL IN COMBINATION WITH MELATONIN TO INDUCE SLEEP

(71) Applicant: Timothy Dale Hewett, Lake Worth, FL (US)

(72) Inventor: Timothy Dale Hewett, Lake Worth, FL (US)

(21) Appl. No.: 17/091,091

(22) Filed: Nov. 6, 2020

Related U.S. Application Data
(60) Provisional application No. 62/932,148, filed on Nov. 7, 2019.
(60) Provisional application No. 63/090,371, filed on Oct. 12, 2020.

(51) Int. Cl. A61K 31/352; A61K 31/05; A61K 31/192; A61K 31/4045

(52) U.S. Cl. CPC A61K 31/352; A61K 31/05; A61K 9/006; A61K 31/4045; A61K 31/192

(57) ABSTRACT

The disclosed embodiments provide chemical compositions and methods for inducing sleep, the compositions comprising active ingredients of (i) major cannabinoids, (ii) minor cannabinoids, and (iii) sleep-regulating compound(s) comprising at least 1 mg of melatonin. The major cannabinoids comprise 15 to 135 mg of CBD, up to 5 mg of Δ9-THC, where the ratio of the CBD to the Δ9-THC is at least 10 to 1. The minor cannabinoids comprise up to 5 mg of CBDv, up to 15 mg of CBN, up to 5 of CBGa, up to 5 mg of CBG, and 0.1 to 5 mg of CBC, where a total amount of the minor cannabinoids is at least 1 mg, and a ratio of the CBD to the minor cannabinoids is at least 5 to 1.

### CROSS-REFERENCE TO RELATED APPLICATIONS

The present U.S. Utility Patent Application claims priority pursuant to 35 U.S.C. § 119(e) to U.S. Provisional Application No. 62/932,148 entitled “CANNABIDIOL AND MELATONIN COMPOSITION”, filed Nov. 7, 2019.

The present U.S. Utility Patent Application claims priority pursuant to 35 U.S.C. § 119(e) to U.S. Provisional Application No. 63/090,371 entitled “PRODUCTS AND METHODS FOR USING CANNABIDIOL IN COMBINATION WITH MELATONIN TO INDUCE SLEEP”, filed Oct. 12, 2020.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### TECHNICAL FIELD

The present invention generally relates to the field of dietary supplements and more particularly relates to supplements for helping sleep sufferers of insomnia, Rapid Eye Movement (REM) sleep behavior disorders, and causes of excessive tiredness (e.g., sleepiness) during the day.

### BACKGROUND

One-third of American adults get less nightly sleep than the recommended seven-hour minimum (Centers for Disease Control and Prevention, 2016). Sleep deprivation can lead to feeling suboptimal throughout the course of a day, even worse, insufficient sleep has been linked to seven of the top 15 causes of death in the U.S. (Kumar Chattu et al., 2018).

Melatonin is a hormone regulating different physiological actions in the brain, ranging from the circadian clock to neurodegeneration. Melatonin is created in the pineal gland. The pineal gland is a pea-sized gland found above the middle brain and functions to control the sleep-wake cycle. Melatonin is responsible for modulating the activities of melatonin in the body. However, factors from stress such as insomnia, alcohol, or too much ambient light before bed can disrupt the creation of Melatonin (Piccirillo, 2007, Rupp, 2006, Jung, Y. & St. Louis, E. K., 2016).

CBD or cannabidiol is a natural chemical compound found in the Cannabis sativa plant. CBD is one of more than 100 different compounds or Phytocannabinoids contained on the flower, and they range and feel and type. In 1992, researchers discovered that, by interacting with receptors in the human ECS, cannabinoids like CBD could deliver unique health benefits to the body in contrast to the cannabinoid Tetrahydrocannabinol (THC). The cannabinoid CBD can counteract the psychoactive effects of THC. Other cannabinoids like Cannabigerol (CBG) and Cannabinol (CBN) can deliver various ranges of effects.

Existing over-the-counter medicines and prescription medicines for overcoming problems associated with sleep have a variety of problems. For instance, the benzodiazepines can cause serious addiction and dependence. Eszopiclone (LUNESTA®) can cause excessive daytime tiredness the day after taking it and can also cause impairment. Antihistamines, such as diphenhydramine (BENADRYL®) can also cause excessive daytime tiredness. Many potential candidates for sleeping medicines avoid these over the counter and prescription medicines due to their negative side effects.

Therefore, a need exists to overcome the problems with the prior art by offering a therapeutic supplement for people living with sleeping disorders (e.g., insomnia, REM sleep behavior disorders) that aids in falling asleep, staying asleep (e.g., getting deep REM sleep), without the negative side effects of the prior art.

### SUMMARY

This Summary is provided to introduce a selection of disclosed concepts in a simplified form that are further described below in the Detailed Description, including the drawings provided. This Summary is neither intended to identify key features or essential features of the claimed subject matter, nor is this Summary intended to be used to limit the claimed subject matter's scope.

The disclosed embodiments are directed to compositions and methods for inducing sleep in a human. It is believed that the compositions described herein exhibit a synergistic effect between at least the cannabidiol (CBD) and melatonin that is more effective than CBD or melatonin on their own. Moreover, it is believed that the minor cannabinoids contribute, at least in part, to the synergistic effect.

Broadly, a single dose of the composition comprises (a) active ingredients and (b) a carrier. The active ingredients generally comprise (i) major cannabinoids, (ii) minor cannabinoids, and (iii) sleep-regulating compound(s). In one embodiment, the major cannabinoids comprise 15 to 135 mg of cannabidiol (CBD) an up to 5 mg of Δ9-tetrahydrocannabinol (Δ9-THC). In one embodiment, the ratio of the CBD to the Δ9-THC is at least 10 to 1. In one embodiment, the minor cannabinoids comprise up to 5 mg of cannabidivarin (CBDv), up to 15 mg of cannabinol (CBN), up to 5 of cannabigerolic acid (CBGa), up to 5 mg of cannabigerol (CBG), and 0.1 to 5 mg of cannabichromene (CBC). In one embodiment, a total amount of the minor cannabinoids is at least 1 mg. In one embodiment, a ratio of the CBD to the minor cannabinoids is at least 5 to 1. In one embodiment, the sleep-regulating compound(s) comprise at least 1 mg of melatonin.

In a more specific embodiment, single dose of composition for inducing sleep in a human comprises active ingredients, wherein the active ingredients comprise: one or more major cannabinoids, one or more minor cannabinoids, and one or more sleep-regulating compound(s). The major cannabinoids comprise 55 to 75 mg of CBD and 1.0 to 4.0 mg of Δ9-THC, where a mass ratio of the CBD to the Δ9-THC is from 30:1 to 50:1. The one or more minor cannabinoids comprise 0.1 to 1.0 mg of CBDv, 0.1 to 1.0 mg of CBN, 0.1 to 1.0 mg of CBG, 2.0 to 4.0 mg of CBC, and up to 1.0 mg of CBGa, where a total amount of the minor cannabinoids is from 2.3 to 6.0 mg, and where a mass ratio of the CBD to the minor cannabinoids is from 10:1 to 30:1. The one or more sleep-regulating compounds comprise 2.0 to 4.0 mg of melatonin, where a mass ratio of the CBD to the melatonin is from 15:1 to 35:1. Lastly, the composition comprises a sublingual carrier for the single dose of the composition, wherein the active ingredients are disposed in the carrier. In one embodiment, the composition is capable of being absorbed by a sublingual cavity of the human.

In another specific embodiment (on a relative mass basis), a composition for inducing sleep in a human, wherein the composition comprises one or more major cannabinoids, one or more minor cannabinoids, and one or more sleep-regulating compound(s). The major cannabinoids comprise 350 to 1300 ppm CBD and 5 to 70 ppm Δ9-THC. The minor cannabinoids comprise 0.5 to 20 ppm CBDv, 0.5 to 20 ppm CBN, 0.5 to 20 ppm CBG,10 to 70 ppm CBC, and up to 20 ppm CBG, where a total amount of the minor cannabinoids is from 15 to 100 ppm. The one or more sleep-regulating compounds comprise 10 to 70 ppm of melatonin. Lastly, the composition comprises a carrier for the composition, with the balance of the composition being the carrier, and the carrier comprising any optional flavorings and any optional colorings.

In one embodiment, a method comprises administering a cannbis composition to a human, the cannbis composition comprising the active ingredients described above.

The foregoing and other features and advantages of the present invention will be apparent from the following more particular description of the invention's preferred embodiments.

### BRIEF DESCRIPTION OF THE DRAWINGS

The accompanying drawings, which are incorporated in and constitute part of this specification, illustrate embodiments of the invention and together with the description, serve to explain the principles of the disclosed embodiments.

FIG. 1a is the chemical structure of the major cannabinoid, cannabidiol (“CBD”);

FIG. 1b is the chemical structure of the major cannabinoid, Δ9-tetrahydrocannabinol (“Δ9-THC”);

FIG. 2a is the chemical structure of the minor cannabinoid, cannabidivarin (“CBDv”);

FIG. 2b is the chemical structure of the minor cannabinoid, cannabinol (“CBN”);

FIG. 2c is the chemical structure of the minor cannabinoid, cannabigerolic acid (“CBGa”);

FIG. 2d is the chemical structure of the minor cannabinoid, cannabigerol (“CBG”);

FIG. 2e is the chemical structure of the minor cannabinoid, cannabichromene (“CBC”); and

FIG. 3 is the chemical structure of melatonin.

### DETAILED DESCRIPTION

The following detailed description refers to the accompanying drawings. While disclosed embodiments may be described, modifications, adaptations, and other implementations are possible. For example, substitutions, additions, or modifications may be made to the compositions described below, and the methods described herein may be modified by substituting reordering or adding additional stages or components to the disclosed methods. Accordingly, the following detailed description does not limit the disclosed embodiments. Instead, the proper scope of the disclosed embodiments is defined by the appended claims.

The disclosed compositions and methods improve over the prior art by providing a composition for inducing sleep that exhibits a synergistic effect between at least the CBD and melatonin. Moreover, the minor cannabinoids may also contribute to the synergistic effect. For instance, one or more of the CBC, CBG, and CBN may contribute to the synergistic effect. In this regard, the compositions described herein may realize an improved combination of properties over the prior art. The improved combination of properties including one or more of: reducing the amount of time to fall asleep, increasing the amount of REM sleep, increasing the total amount of sleep, increasing the feeling of restfulness after a night of sleep, an increase in energy throughout the day, a reduction in daytime tiredness, and a reduction in grogginess in the morning upon waking.

Broadly, the disclosed embodiments provide chemical compositions and methods for inducing sleep, a single dose of the composition comprising (or in some embodiments, consisting essentially of, or consisting of) (a) active ingredients and (b) a carrier. The active ingredients generally comprise (i) major cannabinoids, (ii) minor cannabinoids, and (iii) sleep-regulating compound(s). In one embodiment, the major cannabinoids comprise 15 to 135 mg of cannabidiol (CBD) and up to 5 mg of Δ9-tetrahydrocannabinol (Δ9-THC). In one embodiment, the ratio of the CBD to the Δ9-THC is at least 10 to 1. In one embodiment, the minor cannabinoids comprise up to 5 mg of cannabidivarin (CBDv), up to 15 mg of cannabinol (CBN), up to 5 of cannabigerolic acid (CBGa), up to 5 mg of cannabigerol (CBG), and 0.1 to 5 mg of cannabichromene (CBC). In one embodiment, a total amount of the minor cannabinoids is at least 1 mg. In one embodiment, a mass ratio of the CBD to the minor cannabinoids is at least 5 to 1. In one embodiment, the sleep-regulating compound(s) comprise at least 1 mg of melatonin. These components of the compositions described herein are discussed in greater detail below.

On a relative mass basis, in some embodiments the sum total concentration of the active ingredients may not greater than 1700 ppm. In one embodiment, the sum total concentration of the active ingredients is at least 400 ppm. In another embodiment, the sum total concentration of the active ingredients is at least 500 ppm. In yet another embodiment, the sum total concentration of the active ingredients is at least 650 ppm. In one embodiment, the sum total concentration of the active ingredients is not greater than 1500 ppm. In another embodiment, the sum total concentration of the active ingredients is not greater than 1250 ppm. In yet another embodiment, the sum total concentration of the active ingredients is not greater than 1000 ppm.

In some embodiments, the balance of the composition on a relative mass basis consists of the carrier. The carrier may include any optional flavorings and/or optional colorings. The balance of the composition is calculated as being: 100%—(sum total concentration of active ingredients). As one may appreciate, the balance of the composition may also include trace compounds present in the Cannabis plant, such as phenolic compounds (e.g., flavonoids), fatty acids, chlorophyll, and sterols. In one embodiment, the balance of a composition consists of any optional flavorings, any optional colorings, and trace compounds present in the Cannabis plant.

The compositions described herein may be produced by combining one or more cannbis extracts with the one or more sleep-regulating compound(s), including at least melatonin. A variety of cannbis extracts may be used. A variety of methods for producing cannbis extracts is provided below. Types of cannbis extracts such as full spectrum hemp extract, broad spectrum hemp extract, cannabinoid isolates, cannabinoid concentrates, among others may be used. Cannabinoid isolates (e.g., crystalline isolates) are generally purified to be at least 98 wt. % of a single cannabinoid. Cannabinoid concentrates are generally purified to be at least 80 wt. % of a single cannabinoid, sometimes with a balance of a combination of minor cannabinoids and terpenes. Examples of cannabinoid concentrates include distillates, waxes, crumbles, shatters, and live resins.

The term, “cannbis ” refers to any member of the plant genus Cannabis, including Cannabis sativa, Cannabis indica, and Cannabis ruderalis, including any hybrids thereof

The term, “hemp” takes on substantially the same meaning as defined in the Hemp Farming Act of 2018, meaning hemp is cannbis with less than 0.3 wt. % total of Δ9-THC. The total THC is calculated as the sum of Δ9-THC and Δ9-THCa*0.877. It is appreciated that the definition of “hemp” may change in the future, and hemp may legally contain up to 1.0 wt. % or more of Δ9-THC.

The term “cannbis extracts” refers to the cannabinoids of the cannbis plant that have been extracted and concentrated using one of the many known chemical extraction methods including non-hydrocarbon solvents and hydrocarbon solvents. The term, “hemp extracts” is also used and specifically refers to a cannbis extract of the hemp plant. Non-hydrocarbon solvents such as water, carbon dioxide, isopropyl alcohol, and combinations thereof may be used. Hydrocarbon solvent extracts, such as from butane, propane, hexane, and combinations thereof may be used. Other types of extraction methods, such as physical methods may also be used, such as the dry-sieve method. In one embodiment, an extract is a supercritical CO2 extraction product.

In one embodiment, a composition comprises at least one full spectrum hemp extract. The term, “full spectrum hemp extract” refers to a plant extract from hemp, made using any suitable method and without any modifications to the composition. Full spectrum hemp extract generally has at least some Δ9-THC. It is normal for hemp to have Δ9-THC or any precursors thereof in the hemp plant, and in a full spectrum hemp extract this compound is present. In one embodiment, a full spectrum hemp extract is less than 0.3 wt. % in total of Δ9-THC.

In one embodiment, a composition comprises at least one broad spectrum hemp extract. The term, “broad spectrum hemp extract” refers to a plant extract from hemp, made using any suitable method and with a reduced Δ9-THC content relative to its' parent full spectrum hemp extract. Broad spectrum hemp extract producers may reduce the THC content of the extract using a suitable method, such as chromatography methods known in the art. In one embodiment, a broad-spectrum hemp extract is less than 0.1 wt. % in total of Δ9-THC.

The cannabinoid content of the active ingredients may be produced by combining one or more cannbis extracts. For instance, a full- or broad-spectrum hemp extract may be combined with a cannabinoid isolate, or cannabinoid concentrate to increase the relative concentration of a particular cannabinoid. For instance, a CBD-rich full- or broad-spectrum hemp extract may be combined with a CBC isolate or concentrate to produce a CBC-enriched cannbis extract.

Methods for inducing sleep in a human using a cannbis composition are also disclosed. The methods generally comprise administering to a human a cannbis composition, the cannbis composition comprising the active ingredients (major cannabinoids, minor cannabinoids, and sleep-regulating compound(s)) disposed in a carrier. Compositions suitable for such methods are described in greater detail below.

A preferred method for consuming the compositions described herein generally includes disposing the active ingredients in a sublingually consumable carrier. The sublingually consumable carrier enables a higher bioavailability of the active ingredients. The volume of the single dose is generally from 0.5 milliliters to 1.5 milliliters (“mL”). In one embodiment, the volume a single dose is from 0.75 to 1.25 mL. In one embodiment, the volume of a single dose is approximately 1.0 mL.

i. The Major Cannabinoids

With reference to FIGS. 1a-1b, as noted above, the compositions described herein generally comprise 15 to 135 mg of cannabidiol (CBD). CBD (having chemical abstracts number (“CAS”) 13956-29-1), shown in FIG. la, may prevent the breakdown of the brain's chemical(s) responsible for pain, mood, and mental function. CBD may also block some of the psychoactive effects of Δ9-THC. Other medicinally important effects that CBD may have include anti-anxiety effects, anti-tumor effects, and anti-psychotic effects, among others. In one embodiment, a single dose of a composition includes at least 25 mg of CBD. In another embodiment, a single dose of a composition includes at least 35 mg of CBD. In yet another embodiment, a single dose of a composition includes at least 45 mg of CBD. In another embodiment, a single dose of a composition includes at least 55 mg of CBD. In one embodiment, a single dose of a composition includes not greater than 120 mg of CBD. In another embodiment, a single dose of a composition includes not greater than 105 mg of CBD. In another embodiment, a single dose of a composition includes not greater than 90 mg of CBD. In yet another embodiment, a single dose of a composition includes not greater than 75 mg of CBD. In one embodiment, a single dose of a composition includes 35 to 90 mg of CBD. In another embodiment, a single dose of a composition includes 55 to 75 mg of CBD.

On a relative mass basis, in some embodiments the concentration of CBD in a composition may range from 350 to 1300 ppm. In one embodiment, the CBD concentration in a composition is at least 450 ppm. In another embodiment, the CBD concentration in a composition is at least 575 ppm. In one embodiment, the CBD concentration in a composition is not greater than 1300 ppm. In another embodiment, the CBD concentration in a composition is not greater than 1000 ppm. In yet another embodiment, the CBD concentration in a composition is not greater than 785 ppm.

Generally, the compositions described herein include CBD in an amount such that the mass ratio of CBD to the minor cannabinoids (i.e., the sum total of the minor cannabinoids) is at least 5 to 1. That is, at least five mass units of CBD are present for each mass unit of minor cannabinoids. In one embodiment, a composition has a mass ratio of CBD to minor cannabinoids of at least 7:1. In another embodiment, a composition has a mass ratio of CBD to minor cannabinoids of at least 10:1. In one embodiment, a composition has a mass ratio of CBD to minor cannabinoids of not greater than 40:1. That is, the composition has not greater than 40 mass units of CBD for each mass unit of minor cannabinoids. In another embodiment, a composition has a mass ratio of CBD to minor cannabinoids of not greater than 30:1.

As noted above, the compositions described herein generally comprise up to 5 mg of Δ9-tetrahydrocannabinol (Δ9-THC). Δ9-THC (CAS number 1972-08-3), shown in FIG. lb, is known as being the primary psychoactive cannabinoid in cannbis products, being primarily responsible for the intoxicating effects. Other than the intoxicating effects, Δ9-THC may reduce nausea and act as an appetite stimulant. Furthermore, Δ9-THC may help potentiate the effects of CBD. In one embodiment, a single dose of a composition includes at least 0.5 mg of Δ9-THC. In another embodiment, a single dose of a composition includes at least 1.0 mg of Δ9-THC. In one embodiment, a single dose of a composition includes not greater than 4.0 mg of Δ9-THC.

On a relative mass basis, in some embodiments the concentration of Δ9-THC in a composition may range from 5 to 70 ppm. In one embodiment, the concentration of Δ9-THC in a composition is at least 10 ppm. In one embodiment, the concentration of Δ9-THC in a composition is not greater than 50 ppm. In another embodiment, the concentration of Δ9-THC in a composition is not greater than 40 ppm.

Like many of the cannabinoids, Δ9-THC is produced by the decarboxylation of its' acidic form, Δ9-tetrahydrocannabiolic acid (“Δ9-THCa”). In contrast to Δ9-THC, Δ9-THCa (not illustrated) is not known to be psychoactive. Δ9-THCa may have anti-inflammatory properties and may help slow the progress of degenerative neurological diseases. In one embodiment, a single dose of a composition includes at least 0.1 mg of Δ9-THCa. In another embodiment, a single dose of a composition includes at least 0.5 mg of Δ9-THCa. In yet another embodiment, a single dose of a composition includes at least 1.0 mg of Δ9-THCa. In one embodiment, a single dose of a composition includes not greater than 5.0 mg of Δ9-THCa. In another embodiment, a single dose of a composition includes not greater than 4.0 mg of Δ9-THCa.

On a relative mass basis, in some embodiments the concentration of Δ9-THCa in a composition may range from 0.5 to 20 ppm. In one embodiment, the concentration of Δ9-THCa in a composition is at least 1 ppm. In one embodiment, the concentration of Δ9-THCa in a composition is not greater than 10 ppm.

In one embodiment, a composition comprises CBD and Δ9-THC, where a mass ratio of the CBD to the Δ9-THC is at least 10 to 1 (i.e., at least 10 mass units of CBD for each mass unit of Δ9-THC). In one embodiment, a composition has a mass ratio of CBD to Δ9-THC of at least 20:1. In another embodiment, a composition has a mass ratio of CBD to Δ9-THC of at least 30:1. In one embodiment, a composition has a mass ratio of CBD to Δ9-THC of not greater than 60:1. In another embodiment, a composition has a mass ratio of CBD to Δ9-THC of not greater than 50:1.

The cannabinoids, major and/or minor, may be extracted from at least one cannbis source. For instance, the cannbis source(s) may include hemp. A cannbis source such as hemp may be substantially THC-free, and the resulting compositions produced therefrom may also be substantially THC-free. In one embodiment, a composition is substantially THC-free. In one embodiment, the term, “substantially THC-free” means having less than 0.3 wt. % Δ9-THC. In one embodiment, “substantially THC-free” means than 0.3 wt. % of total THC, the total THC being the sum of Δ9-THC and Δ9-THCa*0.877.

ii. The Minor Cannabinoids

With reference now to FIGS. 2a-2e, as noted above, the compositions described herein generally comprise at least some minor cannabinoids (e.g., at least 0.1 mg of cannabichromene). Minor cannabinoids are cannabinoids that are generally present in significantly lower amounts than the dominant major cannabinoid(s). In the present application, CBD is the dominant major cannabinoid. The minor cannabinoids may have a variety of medicinal properties as discussed in greater detail below. In one embodiment, a single dose of a composition comprises not greater than 40 mg of minor cannabinoids. In another embodiment, a single dose of a composition comprises not greater than 30 mg of minor cannabinoids. In yet another embodiment, a single dose of a composition comprises not greater than 20 mg of minor cannabinoids. In another embodiment, a single dose of a composition comprises not greater than 10 mg of minor cannabinoids. In yet another embodiment, a single dose of a composition comprises not greater than 6.0 mg of minor cannabinoids.

On a relative mass basis, in some embodiments the concentration of the minor cannabinoids in a composition may be up to 100 ppm. In one embodiment, the concentration of the minor cannabinoids in a composition is at least 15 ppm. In another embodiment, the concentration of the minor cannabinoids in a composition is at least 25 ppm. In one embodiment, the concentration of the minor cannabinoids in a composition is not greater than 65 ppm.

Cannabidivarin (CBDv. CAS number 24274-48-4), shown in FIG. 2a, differs from CBD in that CBDv has a propyl group instead of a pentyl group at the 5-position, i.e., this group is shortened by two carbon units in CBDv relative to CBD. CBDv may be useful as an anti-epileptic, anti-nausea and anti-convulsive. In one embodiment, a single dose of a composition comprises up to 5.0 mg of CBDv. In one embodiment, a single dose of a composition comprises not greater than 4.0 mg of CBDv. In another embodiment, a single dose of a composition comprises not greater than 2.5 mg of CBDv. In yet another embodiment, a single dose of a composition comprises not greater than 1.0 mg of CBDv. In one embodiment, a single dose of a composition comprises at least 0.1 mg of CBDv. In another embodiment, a single dose of a composition comprises at least 0.25 mg of CBDv.

On a relative mass basis, in some embodiments the concentration of CBDv in a composition may be up to 20 ppm. In one embodiment, the concentration of CBDv in a composition is at least 0.5 ppm. In another embodiment, the concentration of CBDv in a composition is at least 1.0 ppm. In one embodiment, the concentration of CBDv in a composition is not greater than 10 ppm.

Cannabinol (CBN, CAS number 521-35-7), shown in FIG. 2b, is known to form as THC ages, i.e., CBN is a degradation product of THC. Similar to Δ9-THC and Δ9-THCa, CBN is an appetite stimulant. CBN may also be useful for treating antibiotic-resistant bacterial infections, among other potential uses. In one embodiment, a single dose of a composition comprises up to 15 mg of cannabinol (CBN). In one embodiment, a single dose of a composition comprises not greater than 12 mg of CBN. In another embodiment, a single dose of a composition comprises not greater than 8.0 mg of CBN. In yet another embodiment, a single dose of a composition comprises not greater than 5.0 mg of CBN. In another embodiment, a single dose of a composition comprises not greater than 4.0 mg of CBN. In one embodiment, a single dose of a composition comprises at least 0.1 mg of CBN. In another embodiment, a single dose of a composition comprises at least 0.25 mg of CBN.

On a relative mass basis, in some embodiments the concentration of CBN in a composition may be up to 20 ppm. In one embodiment, the concentration of CBN in a composition is at least 0.5 ppm. In another embodiment, the concentration of CBN in a composition is at least 1.0 ppm. In one embodiment, the concentration of CBN in a composition is not greater than 10 ppm.

Cannabigerolic acid (CBGa, CAS number 25555-57-1), shown in FIG. 2c, is the acidic precursor to cannabigerol (CBG). CBGa may also be considered the parent molecule from which all other cannabinoids are naturally synthesized. CBGa may be useful in a variety of applications, such as in the treatment of inflammatory bowel disease, colon cancer, metabolic disorders, and cardiovascular disease, among others. In one embodiment, a single dose of a composition comprises not greater than 4.0 mg of CBGa. In another embodiment, a single dose of a composition comprises not greater than 2.5 mg of CBGa. In yet another embodiment, a single dose of a composition comprises not greater than 1.0 mg of CBGa. In one embodiment, a single dose of a composition comprises at least 0.1 mg of CBGa. In another embodiment, a single dose of a composition comprises at least 0.25 mg of CBGa.

On a relative mass basis, in some embodiments the concentration of CBGa in a composition may be up to 20 ppm. In one embodiment, the concentration of CBGa in a composition is at least 0.5 ppm. In another embodiment, the concentration of CBGa in a composition is at least 1.0 ppm. In one embodiment, the concentration of CBGa in a composition is not greater than 10 ppm.

Cannabigerol (CBG, CAS number 25654-31-3), shown in FIG. 2d, is the decarboxylated chemical form of CBGa. In one embodiment, a single dose of a composition comprises not greater than 4.0 mg of CBG. In another embodiment, a single dose of a composition comprises not greater than 2.5 mg of CBG. In yet another embodiment, a single dose of a composition comprises not greater than 1.0 mg of CBG. In one embodiment, a single dose of a composition comprises at least 0.1 mg of CBG. In another embodiment, a single dose of a composition comprises at least 0.25 mg of CBG.

On a relative mass basis, in some embodiments the concentration of CBG in a composition may be up to 20 ppm. In one embodiment, the concentration of CBG in a composition is at least 0.5 ppm. In another embodiment, the concentration of CBG in a composition is at least 1.0 ppm. In one embodiment, the concentration of CBG in a composition is not greater than 10 ppm.

Cannabichromene (CBC, CAS number 20675-51-8), shown in FIG. 2e, is another non-psychoactive cannabinoid. CBC may be considered unique in that it may not act on the cannabinoid receptors, but on the TRPVi receptors (vanilloid or capsaicin receptors) and TRPAi receptors. CBC may be useful in a variety of applications, such as an anti-inflammatory, antidepressant, antinociceptive, neuroprotectant, and antibacterial, among others. In one embodiment, a single dose of a composition comprises up to 5.0 mg of CBC. In one embodiment, a single dose of a composition comprises not greater than 4.0 mg of CBC. In one embodiment, a single dose of a composition comprises at least 0.1 mg of CBC. In another embodiment, a single dose of a composition comprises at least 0.5 mg of CBC. In yet another embodiment, a single dose of a composition comprises at least 1.0 mg of CBC. In another embodiment, a single dose of a composition comprises at least 2.0 mg of CBC.

On a relative mass basis, in some embodiments the concentration of CBC in a composition may be up to 70 ppm. In one embodiment, the concentration of CBC in a composition is at least 10 ppm. In another embodiment, the concentration of CBC in a composition is at least 20 ppm. In one embodiment, the concentration of CBG in a composition is not greater than 40 ppm.

Other minor cannabinoids may also be included that are not specifically referenced above. For instance, one or more of the following minor cannabinoids may also be included: Δ8-tetrahydrocannabinol (Δ8-THC), cannabidiorcol (CBDC1), cannabidiol monomethylether (CBDM), cannabidivarinic acid (CBDVa), cannabigerolic acid monomethylether (CBGAM), cannabigerol monomethylether (CBGM), cannabigerovarin (CBGV), cannabigerovarinic acid (CBGVa), cannabinolic acid (CBNa), cannabiorcool (CBN-C1), cannabinol-C2 (CBN-C2), cannabinol-C4 (CBN-C4), cannabinodiol (CBND), cannabinol methylether (CBNM) cannabichromenic acid (CBCa), cannabichromenevarin (CBCV), cannabichromevarinic acid (CBCVa), cannabicyclol (CBL), cannabicyclolic acid (CBLa), cannabicyclovarin (CBLV), cannabitriol (CBT), cannabitriolvarin (CBTV), 10-Ethoxy-9-hydroxy-delta-6a-tetrahydrocannabinol,8,9-Dihydroxy-delta-6a-tetrahydrocannabinol, and cannabinodivarin (CBVD). To the extent that these other minor cannabinoids are included, they are generally included in amounts such that a single dose of the composition comprises not greater than 1.0 mg of each. Furthermore, the sum total of these other cannabinoids may be not greater than 5.0 mg in a single dose of the composition. On a relative mass basis, in some embodiments each other cannabinoid may be present in an amount of not greater than 10 ppm. In some embodiments, the sum total of these other cannabinoids may be not greater than 50 ppm.

References to the major cannabinoids and minor cannabinoids are made on a mass basis. In one embodiment, the major cannabinoids and minor cannabinoids are measured using a THERMO FISHER® ultrahigh performance liquid chromatography (“UHPLC”) system coupled with a diode array detector with methanol as the solvent.

iii. Sleep-Regulating Compounds

With reference now to FIG. 3, as noted above, the compositions described herein generally include at least 1 mg of melatonin (CAS number 73-31-4). In one embodiment, a single dose of a composition comprises not greater than 20 mg of melatonin. In another embodiment, a single dose of a composition comprises not greater than 15 mg of melatonin. In yet another embodiment, a single dose of a composition comprises not greater than 10 mg of melatonin. In another embodiment, a single dose of a composition comprises not greater than 7.0 mg of melatonin. In yet another embodiment, a single dose of a composition comprises not greater than 4.0 mg of melatonin. In one embodiment, a single dose of a composition comprises at least 2.0 mg of melatonin. In one embodiment, a single dose of a composition comprises 2.0 to 4.0 mg of melatonin.

On a relative mass basis, in some embodiments the concentration of melatonin in a composition may be from 10 to 70 ppm. In one embodiment, the concentration of melatonin in a composition is at least 20 ppm. In one embodiment, the concentration of melatonin in a composition is not greater than 40 ppm.

While not being bound by any theory, it is believed that the compositions described herein have a unique mass ratio of CBD to melatonin that provides a synergistic effect. In one embodiment, the mass ratio of the CBD to the melatonin is at least 15:1. In another embodiment, the mass ratio of CBD to melatonin is at least 18:1. In one embodiment, a mass ratio of the CBD to melatonin is not greater than 45:1. In one embodiment, a mass ratio of CBD to melatonin is not greater than 35:1. In another embodiment, a mass ratio of CBD to melatonin is not greater than 26:1.

This application generally relates to compositions having melatonin as the primary, if not the only, sleep-regulating compound. However, other sleep-regulating compounds may additionally be added. For instance, other sleep-regulating compounds that may be used include L-theanine (e.g., up to 100 mg), flowers and extracts of the plant genus Lavandula (lavender), antihistamines (e.g., up to 100 mg), gamma-aminobutyric acid (GABA) (e.g., up to 750 mg), L-tryptophan (e.g., up to 100 mg), magnesium and salts thereof (e.g., up to 100 mg of total magnesium) among others.

iv. Carriers

As noted above, the compositions described herein generally include a carrier, and the active ingredients are disposed within the carrier. A variety of different carriers may be used, including carriers applicable for oral consumption (e.g., sublingual consumption), intravenous consumption, and rectal consumption. Oral consumption is the generally preferred method of consumption.

In one embodiment, the composition is prepared as an oral formulation. Examples of oral formulations include capsules, coated tables, edibles, elixirs (e.g., soft drinks), emulsions, gels, gelcaps, granules, gums, liquids (e.g., oils, hydrophilized aqueous mixtures), pastes, pellets, pills, powders, rapidly-dissolving tablets, sachets, semi-solids, sprays, solutions, suspensions, syrups, tablets, etc. Other oral formulations may also be used and are within the spirit and scope of the invention.

In one embodiment, the composition is prepared as a liquid oral formulation. In one embodiment, the composition is prepared as a lipid-based liquid oral formulation. In one embodiment, the composition is prepared as an alcohol-based liquid oral formulation. In one embodiment, the composition is prepared as a hydrophilized aqueous mixture oral formulation (e.g., where the active ingredients are hydrophilized in cyclodextrin complexes).

Cannabinoids are generally lipophilic compounds that are soluble in lipids (fats), alcohols, and other non-polar organic solvents. Due to their lipophilic nature, the cannabinoids generally have poor solubility in the aqueous fluids of the gastrointestinal tract. In other words, oral formulations of cannabinoids that are absorbed via the gastrointestinal tract have poorer bioavailability than some of their counterparts (e.g., inhalation is known to have better cannabinoid bioavailability). It may be preferred to use a carrier that has high bioavailability, such that the end consumer utilizes as much of the active ingredients as possible.

There are a variety of methods known in the pharmaceutical arts that may be used to increase the bioavailability of lipophilic substances such as cannabinoids. For instance, carriers known in the pharmaceutical arts such as nanoparticles, solid lipid nanoparticles (SLN), complexations with cyclodextrins, polymeric conjugates, permeation enhancers, and surfactants can increase the bioavailability of the active ingredients. Other methods for increasing the bioavailability not mentioned here may also be used, and such methods for increasing the bioavailability are within the spirit and scope of the invention.

As discussed above, carriers that increase the bioavailability of the active ingredients in the gastrointestinal tract itself may be used and may be preferred. However, the method of consumption itself may be modified to increase bioavailability. For instance, sublingual consumption may lead to increased bioavailability of the active ingredients when compared to formulations that are absorbed via the gastrointestinal tract. With sublingual consumption, the active ingredients are directly absorbed by the sublingual vein in the mouth cavity. Thus, in one embodiment, the composition is prepared as an oral formulation for sublingual consumption.

A variety of carriers may be used for sublingual consumption of the compositions described herein. For instance, lipids, alcohols, and other non-polar organic solvents may be used. Polar solvents such as water may also be used, provided that the active ingredients are soluble in, or modified to be soluble in the polar solvent. For instance, water may be used with cannabinoids that have been solubilized via cyclodextrin complexation. In one preferred embodiment, the carrier comprises a medium chain triglyceride oil. In one embodiment, a MCT oil is derived from coconut oil.

The term “medium chain triglyceride” (MCT) “in the present disclosure refers to a class of triglyceride oil that are derived from fatty acids and are usually about 8 to about 12 carbons in length.

The carrier may also include any optional flavoring(s) and/or optional coloring(s). Optional flavoring(s) may provide for a more pleasurable oral consumption experience. Optional coloring(s) may provide for a more pleasurable visual experience when consuming the composition. The optional flavoring(s) and/or coloring(s) may be derived from any suitable compound or combination of compounds. In one embodiment, a flavoring is a mint flavoring. For instance, a mint flavoring may be produced from an extract of one or more of peppermint (Mentha Balsamea Wild) and spearmint (Mentha spicata), among a variety of other mint plants.

Terpenes (e.g., terpenoids) may also be used to provide flavoring and/or coloring to the compositions. The term, “terpene” refers to hydrocarbons of biological origin having carbon skeletons derived from isoprene. Terpenes and terpenoids are commonly subdivided based on the number of isoprene units the compound is derived from: hemiterpenes (C5), monoterpenes (C10), sesquiterpenes (C15), diterpenes (C20), sesterterpenes (C25), triterpenes (C30), sesquarterpenes (C35), tetraterpenes (C40, sometimes called carotenoids), and polyterpenes (greater than 40 carbon skeleton atoms).

Terpenes may be derived from the cannbis extracts used in the compositions described herein. Regardless, for the purposes of this patent application terpenes are considered as being part of the carrier and are not considered active ingredients. Non-limiting examples of terpenes include bisabolol, borneol, caryophyllene, carene, camphene, cineol, citronella, eucalyptol, geraniol, guaiol, humulene, isopropyltoluene, isopulegol, linalool, limonene, menthol, myrcene, nerolidol, ocimene, pinene, phytol, pulegone, terpinene, terpinolene, and thymol, among others.

As noted above, methods for consuming the cannbis compositions are disclosed. The methods generally comprise administering to the human the cannbis composition (e.g., any of the compositions described above). The type of consumption may slightly modify the method of administration to a human. For instance, in one embodiment, the administering comprises orally consuming the cannbis composition. In a more specific embodiment, the administering comprises sublingually administering the cannbis composition. In another embodiment, the administering comprises rectally consuming the cannbis composition. In one embodiment, the cannbis composition is a liquid.

### EXAMPLE 1

A cannbis composition was prepared from a hemp extract. The hemp extract and melatonin were dispersed in a medium chain triglyceride oil, along with mint extract as a flavoring. The hemp extract and melatonin were added in amounts such that 1 milliliter (mL) of the composition provided one dose to a user. The cannbis composition was measured using a THERMO FISHER® ultrahigh performance liquid chromatography (“UHPLC”) system coupled with a diode array detector. Methanol was used as the solvent in the UHPLC system. The resulting chemical analysis is shown in Table 1, below. Melatonin was not measured via the UHPLC system. Rather, the melatonin concentration was calculated based on the amount of melatonin added to the composition per unit of volume.

TABLE 1
Chemical Composition of Example 1 Composition

| | Concentration |
|---|---|
| Analyte | [mg/dose] |
| Δ9-THC | 1.73 |
| Δ8-THC | ND* |
| THCV | ND* |
| THCa | ND* |
| CBDa | ND* |
| CBDV | 0.34 |
| CBN | 0.45 |
| CBGa | ND* |
| CBG | 0.58 |
| CBC | 2.56 |
| CBD | 66.6 |
| Melatonin | 3.00** |
| Total | 72.27 |

*ND = not detected
**Determined by calculation, not measurement

A plurality of individuals (>500 individuals) purchased the composition. Feedback from the users indicated that the sleep supplement was highly effective at inducing a long, quality night of sleep and that users felt well-rested upon waking. None of the users reported any negative side-effects or reported that the composition was ineffective.

It is believed that at least the CBD and melatonin interact synergistically in the human body and are significantly more effective in combination than each compound on their own. It is believed that the minor cannabinoids also contribute, at least in part, to the synergistic effects.

Although the subject matter has been described in language specific to structural features and/or methodological acts, it is to be understood that the subject matter defined in the appended claims is not necessarily limited to the specific features or acts described above. Instead, the specific features and acts described above are disclosed as example forms of implementing the claims.

What is claimed is:

1. A composition for inducing sleep in a human, wherein a single dose of the composition comprises:
(a) active ingredients, wherein the active ingredients comprise:
(i) major cannabinoids comprising:
15 to 135 mg of cannabidiol (CBD);
up to 5.0 mg of Δ9-tetrahydrocannabinol (Δ9-THC);
wherein a mass ratio of the CBD to the Δ9-THC is at least 10:1;
(ii) minor cannabinoids comprising:
up to 5.0 mg of cannabidivarin (CBDv);
up to 15 mg of cannabinol (CBN);
up to 5.0 of cannabigerolic acid (CBGa);
up to 5.0 mg of cannabigerol (CBG);
0. 1 to 5.0 mg of cannabichromene (CBC);
wherein a total amount of the minor cannabinoids is at least 1 mg;
wherein a mass ratio of the CBD to the minor cannabinoids is at least 5 to 1;
(iii) one or more sleep-regulating compounds comprising:
at least 1 mg of melatonin;
wherein a mass ratio of the CBD to the melatonin is at least 15 to 1;
(b) a carrier for the single dose of the composition, wherein the active ingredients are disposed in the carrier.

2. The composition of claim 1, wherein the major cannabinoids comprise at least 25 mg of CBD.

3. The composition of claim 2, wherein the major cannabinoids comprises not greater than 120 mg CBD.

4. The composition of claim 1, wherein the one or more sleep-regulating compounds comprise 2.0 to 4.0 mg of melatonin.

5. The composition of claim 1, wherein the minor cannabinoids comprises at least one of:
at least 0.1 mg of the CBDv;
at least 0.1 mg of the CBN;
at least 0.1 mg of the CBGa; and
at least 0.1 mg of the CBG.

6. The composition of claim 5, wherein the minor cannabinoids comprise not greater than 4.0 mg of the CBDv.

7. The composition of claim 5, wherein the minor cannabinoids comprise not greater than 4.0 mg of the CBN.

8. The composition of claim 5, wherein the minor cannabinoids comprise not greater than 4.0 mg of the CBGa.

9. The composition of claim 5, wherein the minor cannabinoids comprise not greater than 4.0 mg of the CBG.

10. The composition of claim 1, wherein the minor cannabinoids comprise at least 0.5 mg of the CBC, or at least 1.0 mg of the CBC.

11. The composition of claim 1, wherein the minor cannabinoids comprise not greater than 4.0 mg of the CBC.

12. The composition of claim 1, wherein the mass ratio of the CBD to the minor cannabinoids is at least 10:1.

13. The composition of claim 1, wherein the mass ratio of the CBD to the melatonin is at least 18:1.

14. The composition of claim 1, wherein the mass ratio of the CBD to the Δ9-THC is at least 20:1.

15. The composition of claim 1, wherein the single dose comprises up to 1700 ppm of the active ingredients, wherein a balance of the single dose consists of the carrier, the carrier comprising any optional flavoring agents, and any optional coloring agents.

16. The composition of claim 1, wherein the major cannabinoids and minor cannabinoids are extracted from one or more cannbis sources, wherein at least one of the cannbis sources is hemp, and wherein the hemp is substantially THC-free.

17. A composition for inducing sleep in a human, wherein a single dose of the composition comprises:
(a) active ingredients, wherein the active ingredients comprise:
(i) one or more major cannabinoids comprising:
55 to 75 mg of cannabidiol (CBD);
1.0 to 4.0 mg of Δ9-tetrahydrocannabinol (Δ9-THC);
wherein a mass ratio of the CBD to the Δ9-THC is from 30:1 to 50:1;
(ii) one or more minor cannabinoids comprising:
0.1 to 1.0 mg of cannabidivarin (CBDv);
0.1 to 1.0 mg of cannabinol (CBN);
0.1 to 1.0 mg of cannabigerol (CBG);
2.0 to 4.0 mg of cannabichromene (CBC);
up to 1.0 mg of cannabigerolic acid (CBGa);
wherein a total amount of the minor cannabinoids is from 2.3 to 6.0 mg;
wherein a mass ratio of the CBD to the minor cannabinoids is from 10:1 to 30:1;
(iii) one or more sleep-regulating compounds comprising:
2.0 to 4.0 mg of melatonin;
wherein a mass ratio of the CBD to the melatonin is from 15:1 to 35:1;
(b) a sublingual carrier for the single dose of the composition, wherein the active ingredients are disposed in the carrier and capable of being absorbed by a sublingual cavity of the human.

18. The composition of claim 17, wherein the composition consists of elements (a)(i) through (b).

19. A method for inducing sleep in a human using a cannbis composition, the method comprising administering to the human the cannbis composition, wherein the cannbis composition comprises:
(a) the active ingredients, wherein the active ingredients comprise:
(i) major cannabinoids comprising:
15 to 135 mg of cannabidiol (CBD);
up to 5 mg of Δ9-tetrahydrocannabinol (Δ9-THC);
wherein a mass ratio of the CBD to the Δ9-THC is at least 10 to 1;
(ii) minor cannabinoids comprising:
up to 5 mg of cannabidivarin (CBDv);
up to 15 mg of cannabinol (CBN);
up to 5 of cannabigerolic acid (CBGa);
up to 5 mg of cannabigerol (CBG);
0.1 to 5 mg of cannabichromene (CBC);
wherein a total amount of the minor cannabinoids is at least 1 mg;
wherein a mass ratio of the CBD to the minor cannabinoids is at least 5 to 1;
(iii) one or more sleep-regulating compounds comprising:
at least 1 mg of melatonin;
wherein a mass ratio of the CBD to the melatonin is at least 15 to 1; and
(b) a carrier for the single dose of the composition, wherein the active ingredients are disposed in the carrier.

20. The method of claim 19, wherein the administering comprises sublingually consuming the liquid cannbis composition.
